(19) United States
(12) Patent Application Publication
(10) Pub. No.: US 2026/0355459 A1
(43) Pub. Date: Nov. 12, 2026

(54) MICROCATHETER DEVICE WITH NON-LINEAR BENDING STIFFNESS

(71) Applicant: SCIENTIA VASCULAR, INC., West Valley City, UT (US)

(72) Inventors: Richard F. Maggio, Park City, UT (US); Edward J. Snyder, Park City, UT (US)

(21) Appl. No.: 19/747,147

(22) Filed: Jul. 20, 2026

Related U.S. Application Data
(63) Continuation of application No. 17/901,821, filed on Sep. 1, 2022.
(60) Provisional application No. 63/240,845, filed on Sep. 3, 2021.
(60) Provisional application No. 63/271,114, filed on Oct. 22, 2021.

(51) Int. Cl. A61M 25/00

(52) U.S. Cl. CPC A61M 25/0054; A61M 25/0023; A61M 25/008; A61M 2025/0042

(57) ABSTRACT

Disclosed are microcatheter devices with features that provide effective axial response, good distribution of bending forces, and a smooth bending stiffness profile that minimizes abrupt changes in stiffness. A catheter device includes a microfabricated inner shaft having a plurality of gaps, and an outer member comprising a polymer material disposed within the gaps. The catheter device provides non-linear bending stiffness such that bending becomes more difficult as the bend angle increases.

### CROSS-REFERENCE TO RELATED APPLICATIONS

This application is a continuation of U.S. application Ser. No. 17/901,821, filed Sep. 1, 2022 and titled “Microcatheter Device with Non-Linear Bending Stiffness,” which claims priority to and the benefit of U.S. Provisional Application No. 63/271,114, filed Oct. 22, 2021 and titled “Intravascular Guidewire and Microcatheter System,” and to U.S. Provisional Application No. 63/240,845, filed on Sep. 3, 2021 and titled “Microcatheter Device with Non-Linear Bending Stiffness,” each of which is incorporated herein by this reference in its entirety.

### BACKGROUND

Interventional devices such as guidewires and catheters are frequently utilized in the medical field to perform delicate procedures deep within the human body. Typically, a catheter is inserted into a patient's femoral, radial, carotid, or jugular vessel and navigated through the patient's vasculature to the heart, brain, or other targeted anatomy over a guidewire. Once in place, the catheter can be used to deliver drugs, stents, embolic devices, radiopaque dyes, or other devices or substances for treating the patient in a desired manner.

In many applications, such an interventional device must be navigated through the tortuous bends and curves of a vasculature passageway to arrive at the targeted anatomy. Such an interventional device requires sufficient flexibility, particularly closer to its distal end, to navigate such tortuous pathways. However, other design aspects must also be considered. For example, the interventional device must also be able to provide sufficient torquability (i.e., the ability to transmit torque applied at the proximal end all the way to the distal end), pushability (i.e., the ability to transmit axial push to the distal end rather than bending and binding intermediate portions), and structural integrity for performing intended medical functions.

It is desirable for catheter devices to have good axial response. In other words, when the user moves the proximal end of the device, he/she expects the distal end to move the same distance. Often, however, as the device is positioned within the bends and curves of the vasculature, much of the axial movement is used to push intermediate portions of the device into the walls of the curved portions of the vasculature rather than actually advancing the distal end forward. This lack of correspondence between the amount of axial push provided by the user at the proximal end and the resulting forward movement of the distal end makes navigation more difficult and less tactilely intuitive.

In addition, conventional catheter devices utilize multiple different materials to provide a gradient in bending stiffness from proximal end to distal end. However, whenever there is a transition between materials of differing stiffness, the bending, axial, and torsional stiffness profiles of the device includes an abrupt step change. Such abrupt changes in bending stiffness are undesirable because they can concentrate mechanical stresses at particular locations, cause kink points, disrupt the smooth movement and bending of the device, and complicate navigation in tortuous vasculature.

Accordingly, there exist several limitations in the field of catheter devices, and there is an ongoing need, for example, for devices that improve axial and/or torsional response, the distribution of bending forces, and/or are capable of providing a smooth bending stiffness profile.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an overview of a catheter system, including a catheter and hub;

FIG. 2 illustrates a detailed view showing various sections of the catheter of the catheter system;

FIGS. 3A through 3C illustrate examples of a one-beam section, two-beam section, and three-beam section, respectively, that may be included in a microfabricated shaft used in the catheter system described herein;

FIG. 4 illustrates a detailed view of a distal section of the catheter;

FIGS. 5A through 5D are photographs illustrating differences in axial response that can occur within a vessel (artificial vessel shown), with FIGS. 5A and 5B showing the axial response of a conventional catheter and FIGS. 5C and 5D showing the improved axial response of a catheter according to the present disclosure;

FIG. 6 schematically illustrates a section of the catheter during bending, showing how the catheter is configured to distribute bending, torsional, and axial forces in tortuous anatomy;

FIGS. 7A and 7B illustrate that the microfabricated shaft is configured to compensate for step changes in stiffness in the outer polymer layer due to transition from one polymer to another;

FIGS. 8A and 8B compare a bending stiffness profile of a catheter device according to the present disclosure (labelled “Plato 17”) with bending stiffness profiles of various conventional catheter devices, with FIG. 8A showing bending stiffness up to 60 cm from the distal tip and FIG. 8B showing bending stiffness up to 15 cm from the distal tip; and

FIG. 9 compares outer diameters along distal lengths of a catheter device according to the present disclosure (labelled “Plato 17”) to various conventional catheter devices.

### DETAILED DESCRIPTION

### Overview of Example Catheter Device

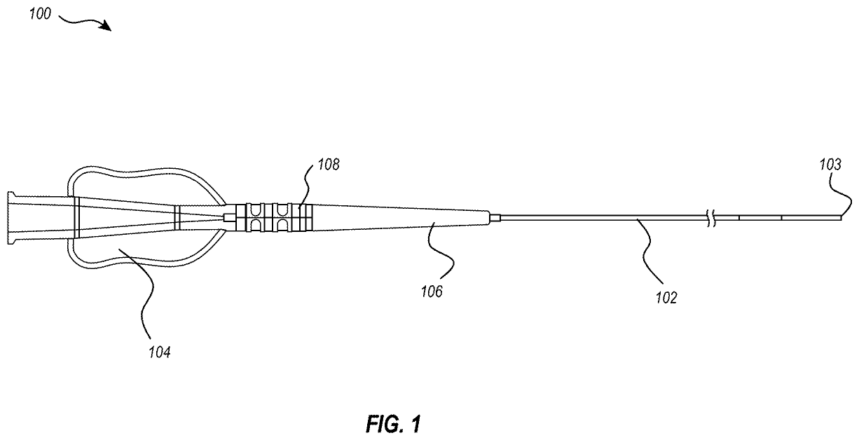

FIG. 1 is an overview of an exemplary catheter device 100 that includes features, described in more detail below, that provide one or more of improved axial responsiveness, improved distribution of bending forces, and/or a smooth device bending stiffness profile.

The catheter device 100 includes a catheter 102 connected to a hub 104 at a proximal end and extending therefrom to a distal end 103. The catheter 102 may be coupled to the hub 104 using adhesive, a friction fit, through insertion molding, and/or other appropriate attachment means. A strain-relief member 106 is also disposed over the proximal section of the catheter 102 near the hub 104. The strain-relief member 106 has an outer diameter that substantially matches the adjacent section of the hub 104. The strain-relief member 106 extends for a distance from the hub 104 with a substantially constant outer diameter before tapering distally to the end where the catheter 102 emerges and extends farther distally. The strain-relief member 106 may include a groove pattern 108, disposed at the section of substantially constant outer diameter, that functions to provide additional flexibility to the strain-relief member 106 and/or to provide surface features for enhancing user grip and tactile engagement.

The working length of the catheter 102 (i.e., the distance between the distal end of the strain-relief (106) and the catheter (102) distal end (103) may vary according to particular application needs. As an example, the catheter 102 may have a working length of about 50 cm to about 200 cm, though shorter or longer lengths may be utilized where appropriate. The catheter size (typically referring to the inner diameter/lumen size) may also vary according to particular application needs. Examples include 0.010 inches, 0.013 inches, 0.017 inches, 0.021 inches, 0.027 inches, 0.030 inches, 0.035 inches, 0.038 inches, 0.045 inches, 0.065 inches, 0.085 inches, 0.100 inches, or a range including any two of the foregoing values as endpoints. The inside diameter of the catheter can taper from a smaller distal portion to a larger proximal portion. Smaller or larger sizes may be utilized in some applications as appropriate.

Although the distal section of the catheter 102 is shown in this example as having a straight shape, other embodiments may include a shaped distal tip. For example, the distal section of the catheter 102 may have an angled shape, a curved shape (e.g., 45 degree angle, 90 degree angle, J shape, etc.), a compound curved shape, or other appropriate angled or bent shape as known in the art.

The catheter device 100 described herein may be utilized for a variety of interventional applications, most commonly in cardiovascular, peripheral vascular, and neurovascular interventional procedures. Examples include accessing distal anatomy, crossing vessel lesions or blood clots, ischemic treatments, delivering therapeutic agents (e.g., embolic coils or other embolic agents), injecting diagnostic agents (e.g., contrast media or saline), retrieval applications, aspiration applications, or other applications where microcatheter use is beneficial.

Internal features of the catheter 102 are described in greater detail below. The outer surface of the catheter 102 may be coated with an appropriate coating material, such as a hydrophilic coating, to make the surface more lubricious. The coating material may cover substantially all of the working length of the catheter 102, or a portion thereof. For example, the coating material may be applied to the distal-most 30% to 80% of the working length of the catheter 102.

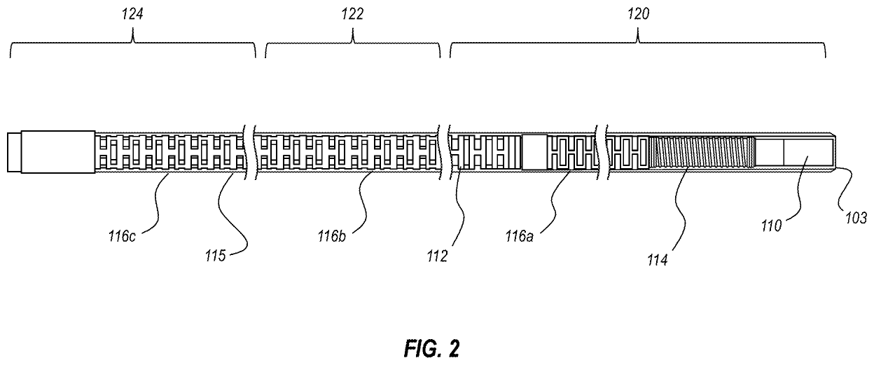

FIG. 2 illustrates a detailed view of the catheter 102, better showing some of the internal components and different longitudinal sections of the catheter 102. As shown, the catheter 102 includes an inner liner 110 that defines the inner lumen of the device. The liner 110 may be formed from polytetrafluoroethylene (PTFE) and/or other appropriate polymer. A coil 114 is positioned on the wire near the distal end 103. The coil 114 is attached to or positioned next to a microfabricated shaft 112 (also referred to herein as “inner shaft”) that extends proximally therefrom. An outer member 115, formed from one or more polymer materials, is typically heat shrink laminated over and through the coil 114 and shaft 112, encasing both while also attaching to the liner.

In one embodiment, the coil 114 is formed from stainless steel and the shaft 112 is formed from nitinol. These materials, when used in combination with other features described herein, have been found to provide effective axial response, effective distribution of bending forces, and a smooth bending stiffness profile. Other embodiments may utilize one or more different materials for the coil 114, the shaft 112, or both. In some embodiments, for example, the shaft 112 may include other superelastic alloys and/or one or more polymers such as a polyether ether ketone (PEEK) or other polyaryl ether ketone (PAEK). In some embodiments, the coil 114 may include a superelastic alloy such as nitinol, one or more other metals, alloys, or polymers.

The catheter 102 is configured so that the overall bending stiffness profile transitions from higher stiffness (and less bending flexibility) at the proximal sections to lower stiffness (and greater bending flexibility) at the distal sections. In most applications, it is desirable to give the proximal sections of the device relatively high axial, torsional, and bending stiffness so they can provide good combination of flexibility, pushability, and torquability. Distal sections, however, are often navigated through tortuous vasculature and are thus preferably relatively more flexible in bending. This stiffness profile gradient can be created by adjusting certain features of the microfabricated shaft 112 and/or by utilizing different polymer materials to coat and embed the shaft 112 in the outer member 115. As explained in more detail below, in some embodiments, the microcatheter 112 and the outer member 115 are configured to work together to provide an overall stiffness profile that minimizes abrupt changes in stiffness and provides smooth stiffness transitions.

The illustrated embodiment includes a distal section 120, an intermediate section 122, and a proximal section 124. In the distal section 120, the outer member 115 is formed from a first polymer material 116a. In the intermediate section 122, the outer member 115 is formed from a second polymer material 116b. In the proximal section 124, the outer member 115 is formed from a third polymer material 116c. The polymer materials 116a, 116b, and 116c have different hardness and thus affect the stiffness of their respective sections differently. The second polymer material 116b has a higher hardness than the first polymer material 116a, and the third polymer material 116c has a higher hardness than the second polymer material 116b. Some embodiments may include more than three polymer materials. In such embodiments, as with the illustrated embodiment, the polymer materials may have progressively higher hardness moving from one polymer material to the next in the proximal direction.

As one example of a set of polymer materials found to be effective when utilized together, the first polymer material 116a may have a Shore D hardness of about 20 to about 30, the second polymer material 116b may have a Shore D hardness of about 30 to about 50, and the third polymer material 116c may have a Shore D hardness of about 50 to about 80. Other embodiments may vary these values as desired such as softer in the distal portion, but the foregoing values have been found to be particularly effective. The polymer materials 116a, 116b, and 116c may be formed from independently from appropriate polymers such as polyether block amide (PEBA) polymers and can range in polymer durometers from Shore A hardness of about 10 to Shore D hardness of about 100.

The shaft 112 also includes features that provide variable bending stiffness. As shown, the shaft 112 is a tube structure that includes a series of microfabricated cuts. The cuts form axially extending “beams” that connect successive circumferentially extending “rings”. These cut patterns can be varied to adjust the bending stiffness of the shaft 112. For example, the bending stiffness can be attuned by adjusting the number of beams that reside between each pair of adjacent rings. A “two-beam section”, such as shown in the distal section 120, includes two beams between each pair of adjacent rings. A “three-beam section”, such as shown in the intermediate section 122 and proximal section 124, includes three beams between each pair of adjacent rings. All else being equal (shaft material, cut depth, cut width, cut spacing), a three-beam section has greater bending stiffness than a two-beam section. A “one-beam section” with a single beam connecting adjacent rings may also be utilized, and will have even less bending stiffness than a two-beam section, all else being equal. A “four-beam section” and/or section having greater than four beams may also be utilized, and will accordingly provide greater bending stiffness as the number of beams between each pair of adjacent rings is increased.

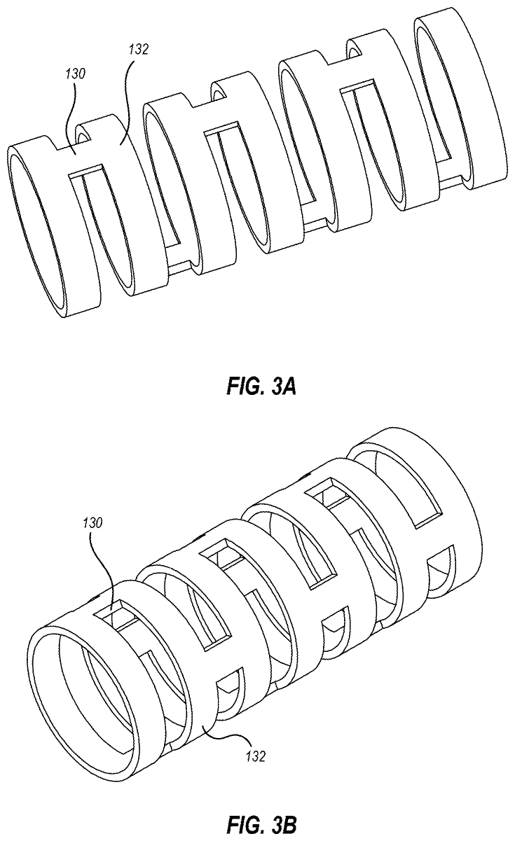

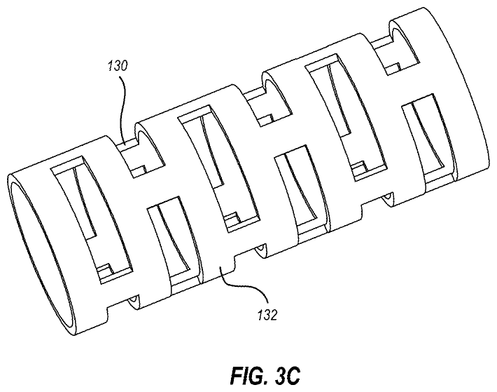

FIGS. 3A through 3C illustrate examples of a one-beam section, two-beam section, and three-beam section, respectively, showing exemplary arrangements of beams 130 and rings 132 in such sections. The beams can be configured in a variety of arrangements depending on the angular offset (or lack thereof) between successive sets of beams and/or how frequently the angular offset is applied (e.g., after each ring or after two or more rings). The one-beam section of FIG. 3A, for example, includes a 180 degree angular offset from one beam to the next, the two-beam section of FIG. 3B includes a 90 degree offset from one beam pair to the next, and the three-beam section of FIG. 3C includes a 120 degree angular offset from one set of beams to the next. While these types of offsets are beneficial, they are also associated with preferred bending planes, and other arrangements may be provided to minimize or eliminate preferred bending planes. Examples include a helical arrangement, a distributed arrangement, an imperfect ramp arrangement, and a sawtooth arrangement. Additional details regarding beam arrangements that may be utilized in the presently disclosed shaft 102 are provided in United States Patent Application No. 2020/0121308, which is incorporated herein by this reference in its entirety.

In addition to adjusting the number of beams disposed between rings, the bending flexibility of the shaft 112 may be controlled by adjusting the depth of cuts, the width of cuts, and/or the spacing of cuts. Typically, the width of cuts is set at a given value (e.g., corresponding to a cutting blade size), and it is easier to adjust cut depth and/or cut spacing during manufacture in order to provide the desired control over the bending stiffness profile. All else being equal, as ring width is reduced (i.e., cut spacing is reduced), cut width is increased, and/or beam width is reduced (i.e., cut depth is increased), the resulting bending stiffness is reduced. In the illustrated embodiment, the spacing between cuts in the three-beam section (which is coincident with the intermediate section 122 and proximal section 124) is progressively reduced as it gets closer to the distal section 120. Similarly, the two-beam section (which is coincident with the distal section 120) begins with larger spaces between cuts and progresses to less space between cuts as it gets closer to the coil 114 and the distal end 103. Preferably, transitions between different geometries (e.g., three-beam to two-beam) are configured so that bending stiffness is the same or similar across the transition of these sections. The shaft 112 thus provides a stiffness gradient by way of transitioning from a three-beam section to a two-beam section, and also within the respective sections by way of transitioning from cuts that are more spaced apart to cuts that are relatively less spaced apart.

At the distal end of the two-beam section, the cut pattern is configured with relatively high flexibility in order to provide a smooth transition to the high flexibility of the coil 114. In some embodiments, the coil 114 is omitted and replaced by more of the two-beam section (or alternatively, a one-beam section) that extends to a position at or near the distal end 103.

The lengths of the sections 120, 122, and 124 may be varied according to particular application needs or preferences. In one embodiment, the distal section 120 may have a length of about 5 cm to about 40 cm, and the intermediate section 122 may have a length of about 10 cm to about 50 cm, with the proximal section 124 taking up the remainder of the working length of the catheter 102. The illustrated embodiment shows that the shaft 112 transitions from a three-beam configuration to a two-beam configuration at the transition of the intermediate section 122 to the distal section 120. However, the transitions of the shaft 112 need not necessarily correspond to the transitions of polymer material that define the separate sections 120, 122, and 124. As explained in more detail below, the shaft 112 and outer member 115 are configured together to compensate for and minimize abrupt stiffness changes, and in some instances, this may involve shaft transition zones that do not overlap completely with polymer transitions of the outer member 115.

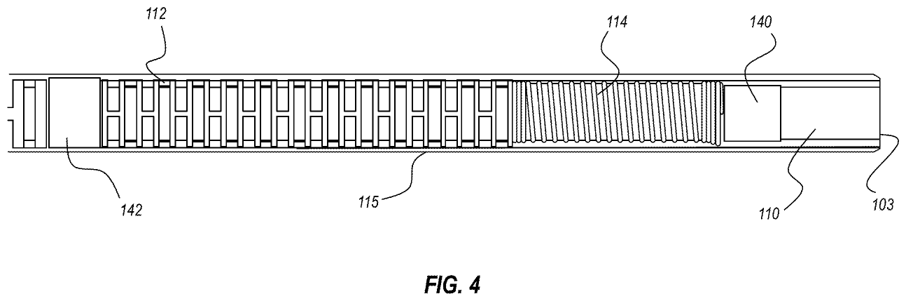

FIG. 4 illustrates a detailed view of the distal section 120 of the catheter 102, better showing certain distal features such as the liner 110, distal radiopaque marker band 140, coil 114, shaft 112, and proximal radiopaque marker band 142. The marker bands 140 and 142 are formed from a material more radiopaque than stainless steel. Examples include platinum, iridium, tungsten, other highly radiopaque metals, and alloys thereof. The distal marker band 140 provides an indication of the location of the distal end 103 of the catheter 102, whereas the proximal marker band 142 is offset by a predetermined length (e.g., 2 to 5 centimeters, or about 3 centimeters) to assist in proper positioning of detachable embolic coils or other components deployed through the catheter 102.

The shaft 112 may include a circumferential groove at the position where the proximal marker band 142 is placed. This groove can receive the marker band 142 such that the outer surface of the marker band 142 does not extend excessively beyond the outer diameter of the shaft 112. Once covered by the outer member 115, the outer diameter of the device over the proximal marker band 142 remains substantially flush.

In the illustrated embodiment, the coil 114 is variably pitched. Each end of the coil 114 includes a region of narrowed pitch that provides more improved transitions in bending stiffness from one geometry to another such as where the coil transitions to a microfabricated tube. As an example, the coil 114 may have a length of about 1 cm to about 3 cm. As shown, a portion of the liner 110 may extend a distance distally from the coil 114 and distal marker coil 140. This distance may vary from about 0.2 mm to about 2 mm, for example.

### Bending Force Distribution

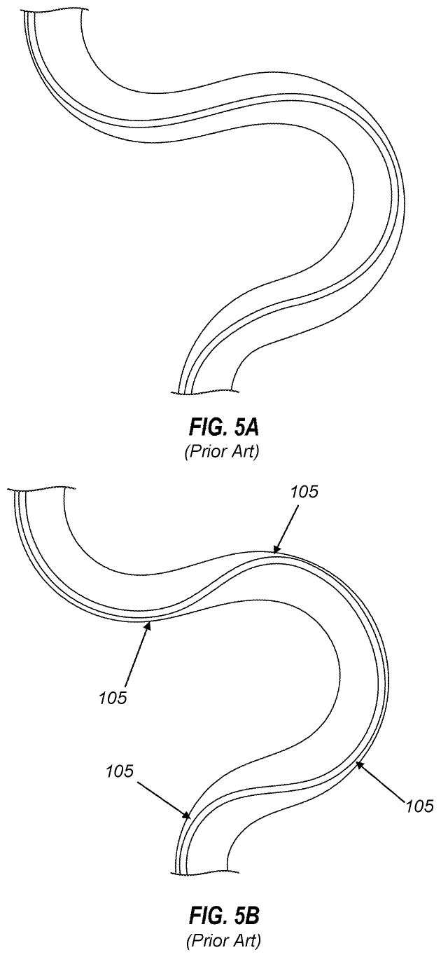

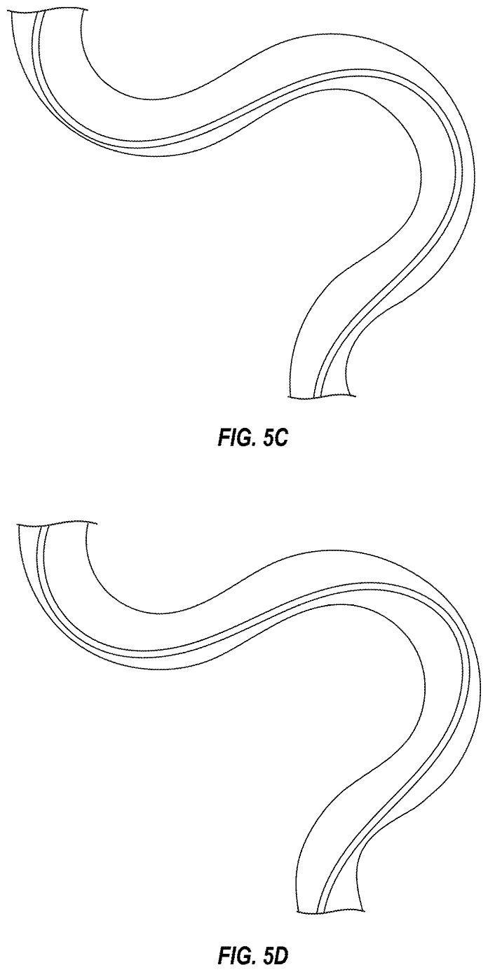

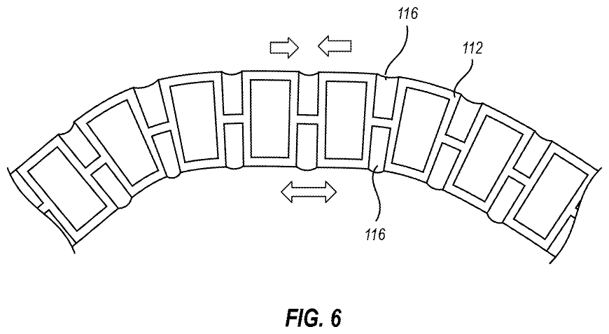

The catheter devices described herein include features that effectively distribute bending forces and thereby provide improved axial response in use. FIGS. 5A and 5B illustrate a common limitation in navigating a conventional catheter (an Excelsior SL-10 shown in this example) through an artificial vasculature construct. As the catheter approaches a bend in a vessel, a certain length of the catheter extends around the bend (initial position in FIG. 5A). Upon further pushing, the initial axial movement is taken up in pushing the catheter against the walls of the vessel to fill the curves (after push position in FIG. 5B; see contact points 105 indicated in FIG. 5B) before any continued pushing results in actually advancing the distal tip of the catheter through the vasculature. This reduced correspondence between the amount of axial push provided by the user at the proximal end and the resulting forward movement of the distal end makes navigation more difficult and less tactilely intuitive.

In contrast to the response of conventional catheters as in FIGS. 5A and 5B, FIGS. 5C and 5D show navigation of the bend of the vessel using the catheter 102 as described herein. As shown, from the “initial” position (FIG. 5C) to the after-push position (FIG. 5D), less of the axial movement is taken up in filling the curve of the vessel, and more of the axial movement is thus transferred to actual movement of the distal end of the catheter 102. This function stems from the improved ability to distribute bending forces along the length of the catheter 102. By better distributing the bending forces, the catheter 102 better resists bending at any one particular location and can thereby better transfer proximal axial movement to the distal end of the device.

FIG. 6 further illustrates the ability of the catheter 102 to effectively distribute bending forces. FIG. 6 illustrates a portion of the shaft 112 during bending. The polymer material 116 fills the gaps between beams and rings of the shaft 112. For ease of viewing, the polymer material 116 is shown in discrete sections within each of the gaps of the shaft 112. In most cases, the polymer material 116 will fill the gaps, fuse with the liner 110, and also extend over the outer surface of the shaft 112 to fully encapsulate and embed the shaft 112.

During bending of the shaft 112, the shaft structure locally resists more bending stresses and distributes this stress to adjacent portions of the structure more effectively as compared to a coil or braid which are less likely to distribute bending stresses and more likely to kink. Additionally, the polymer material 116 effectively functions as a series of dampers each positioned between adjacent rings of the shaft 112. On the inner side of the bend, the polymer material 116 is compressed, and therefore provides a counteracting force that pushes outward against the rings and resists further bending. Similarly, on the outer side of the bend, the polymer material 116 is placed in tension, and therefore provides a counteracting force that pulls the rings inward and resists further bending. The bending stiffness of the catheter 102 is non-linear because it becomes increasingly resistant to bending, in a non-linear manner, as the bend angle is increased.

During bending, a conventional catheter will begin to bend at the apex and the cross-sectional shape of the catheter may tend to “ovalize”. Once ovalization begins, resistance to bending decreases and it therefore becomes increasingly easier to bend with continued application of bending forces. In contrast, in the disclosed catheter 102, the bending resistance provided by the action of the microfabricated structure and polymer material 116 against the shaft 112 tends to distribute bending forces along the axial length of the catheter 102 and avoid ovalization and concentration of bending forces at a particular kink point. For example, the bending resistance will tend to spread a bend out to a larger radius of curvature rather than concentrating the bend at specific point thereby creating a kink location. The bending resistance provided by the catheter structure can therefore provide enhanced axial responsiveness (as illustrated by FIGS. 5C and 5D) and also enhanced protection against mechanical fatigue caused by bending stresses.

### Smoothing of Bending Stiffness Profiles

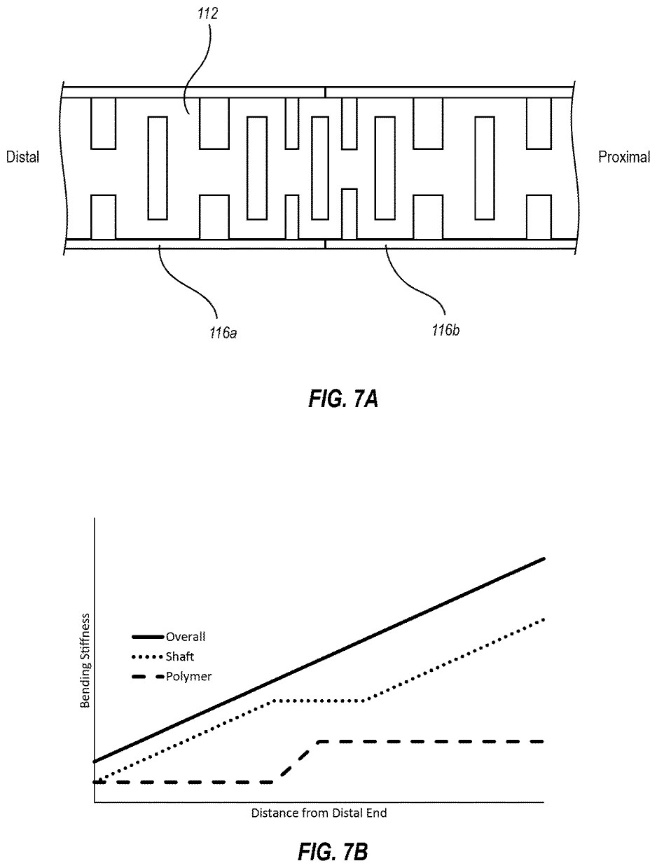

FIGS. 7A and 7B illustrate that the microfabricated shaft may be configured to compensate for step changes in stiffness in the outer member due to transition from one polymer to another. FIG. 7A illustrates a portion of the catheter 102 where the first polymer material 116a meets the second polymer material 116b. As shown in FIG. 7B, this is associated with an abrupt step change in the bending stiffness of the polymer layer of the outer member. Such abrupt changes in bending stiffness are undesirable because they can concentrate mechanical stresses, cause kink points, disrupt the smooth movement and bending of the device, and complicate navigation in tortuous vasculature.

To compensate for this step change, the shaft is configured such that the bending stiffness changes complement and compensate for the abrupt change in bending stiffness of the polymer outer member. As a result, the overall bending stiffness of the catheter remains relatively smooth across the transition from the first polymer 116a to the second polymer 116b. Similar configurations can be utilized at other polymer transition zones to minimize and smooth out abrupt step changes in bending stiffness. The shaft 112 can be configured to compensate for the step change in a variety of ways. In the example of FIG. 7A, because the bending stiffness of the second polymer 116b is higher than the first polymer 116a, the configuration of the cuts/gaps of the shaft 112 remain constant or are narrowed for a short distance across the transition before spreading out again to generally increase shaft bending stiffness while moving further proximally. Other means of adjusting the bending stiffness of the shaft 112 as described herein (e.g., adjusting the number of beams and/or cut depth) may additionally or alternatively be used to achieve the result of smoothing the overall bending stiffness profile.

Configuring the shaft 112 to compensate for abrupt bending stiffness changes of the polymer outer member 115 beneficially avoids the complications of other conventional approaches to smoothing out such transitions. Prior approaches rely on complicated splicing arrangements or polymer co-extrusion and mixing techniques. These add layers of extra complication to the manufacturing process and may still only marginally resolve the abruptness of the transition. Other approaches utilize different diameters of polymer tubing and diameter gradients to compensate for transitions between polymer types. However, these approaches result in an uneven outer diameter or add the requirement to somehow manage this by overlaying even more material.

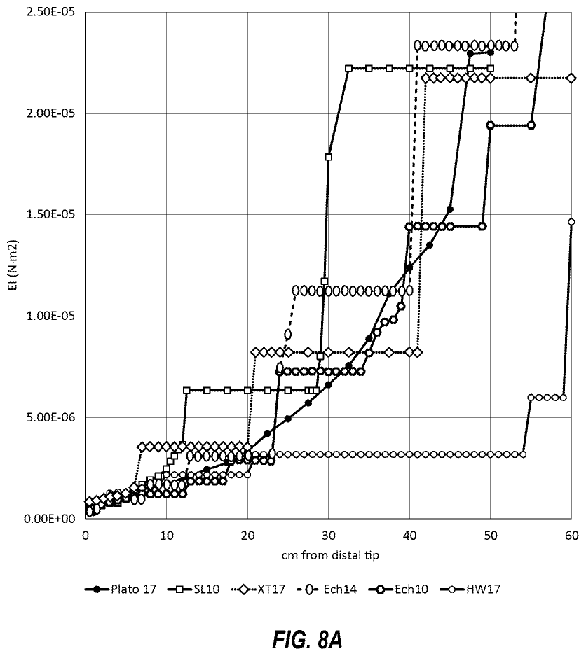

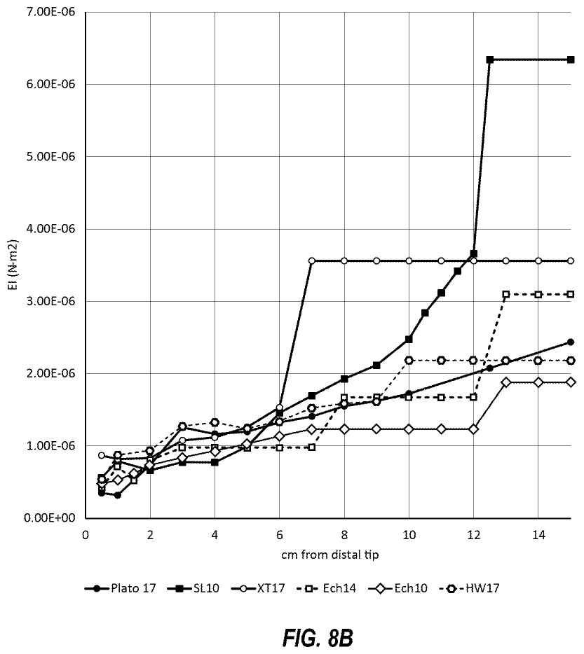

The smoothing features described herein enable the manufacture of microcatheters having improved bending stiffness profiles as compared to conventional microcatheters. FIGS. 8A and 8B illustrate the results of testing comparing the bending stiffness profile of a catheter made according to the present disclosure (labelled “Plato 17”) to the bending stiffness profiles of several conventional microcatheters. In the Figures, “SL10” refers to an Excelsior SL-10 (sold by Stryker Neurovascular), “XT17” refers to an Excelsior XT-17 (sold by Stryker Neurovascular), “Ech14” refers to an Echelon 14 (sold by Medtronic), “Ech10” refers to an Echelon 10 (sold by Medtronic), and “HW17” refers to a Headway 17 (sold by MicroVention Terumo). FIG. 8A illustrates bending stiffness profiles over the distal 50 to 60 cm of the devices, FIG. 8B provides a closer view of the bending stiffness profiles over the distal 15 cm of the devices. In the Figures, the Plato 17 data represents an average of 5 replicates, the SL10 data represents an average of 3 replicates, the XT17 represents an average of 2 replicates, the Ech14 represents an average of 2 replicates, the Ech10 represents an average of 3 replicates, and the HW17 represents an average of 2 replicates. As shown, the catheter corresponding to the present disclosure provides a smoother profile with less abrupt changes in bending stiffness.

Table 1 presents the data of FIGS. 8A and 8B by listing different distal section sizes and providing the highest measured “slope” within that section. The “slope” represents the change in bending stiffness (N·m²) over the distance between measured data points (cm). As evident from the data points in FIGS. 8A and 8B, note that measurements were taken at increments of 0.5 cm to 2.5 cm, usually every 1 cm with smaller increments at regions where a clear polymer transition was evident and larger increments once reaching about 15 to 20 cm from the distal end. The slope therefore provides an indication of the abruptness of bending stiffness changes across the given section of the catheter.

TABLE 1
Stiffness Changes of Various Microcatheters Across Different Distal
Section Sizes.

| | |
|---|---|
| Section Measured from | Highest Measured “Slope” Within Section |
| Distal End (cm) | ((N · m²)/cm) |

Plato 17

| | |
|---|---|
| 0-15 | 5.20e⁻⁷ |
| 0-35 | 5.20e⁻⁷ |
| 0-50 | 8.91e⁻⁷ |

SL-10

| | |
|---|---|
| 0-15 | 5.37e⁻⁶ |
| 0-35 | 1.22e⁻⁵ |
| 0-50 | 1.22e⁻⁵ |

XT-17

| | |
|---|---|
| 0-15 | 2.03e⁻⁶ |
| 0-35 | 4.65e⁻⁶ |
| 0-50 | 1.35e⁻⁵ |

Echelon-14

| | |
|---|---|
| 0-15 | 1.42e⁻⁶ |
| 0-35 | 4.32e⁻⁶ |
| 0-50 | 1.21e⁻⁵ |

Echelon-10

| | |
|---|---|
| 0-15 | 6.46e⁻⁷ |
| 0-35 | 4.39e⁻⁶ |
| 0-50 | 4.97e⁻⁶ |

Headway-17

| | |
|---|---|
| 0-15 | 7.05e⁻⁷ |
| 0-35 | 1.00e⁻⁶ |
| 0-50 | 1.00e⁻⁶ |

As shown, the Plato 17 has the lowest measured slope across the distal 15 cm section, across the distal 35 cm section, and across the distal 50 cm section. Based on the data of FIGS. 8A and 8B, as further disclosed in Table 1, in some embodiments a catheter device as described herein has a bending stiffness slope ((N·m²)/cm)) of no more than about 6.0×10⁻⁷ for a distal 15 cm section, no more than about 9.0×10⁻⁷ for a distal 35 cm section, and/or no more than about 9.0×10⁻⁷ for a distal 50 cm section. While the Plato 17 device configured according to the present disclosure achieved each of the foregoing features, none of the other prior catheter devices tested were able to do so.

In some embodiments, at least a portion of the distal 35 cm of the catheter device has a bending stiffness of 5×10⁻⁶ N·m² or greater. In some embodiments, in addition to the foregoing stiffness minimum, a catheter device as described herein has a bending stiffness slope ((N·m²)/cm)) of no more than about 4.0×10⁻⁶ for a distal 35 cm section and/or no more than about 4.5×10⁻⁶ for a distal 50 cm section. As shown in FIGS. 8A and 8B and Table 1, while the Plato 17 device meets these requirements, the Headway-17 catheter does not meet the minimum bending stiffness requirement, and none of the other tested catheters meet the slope requirements.

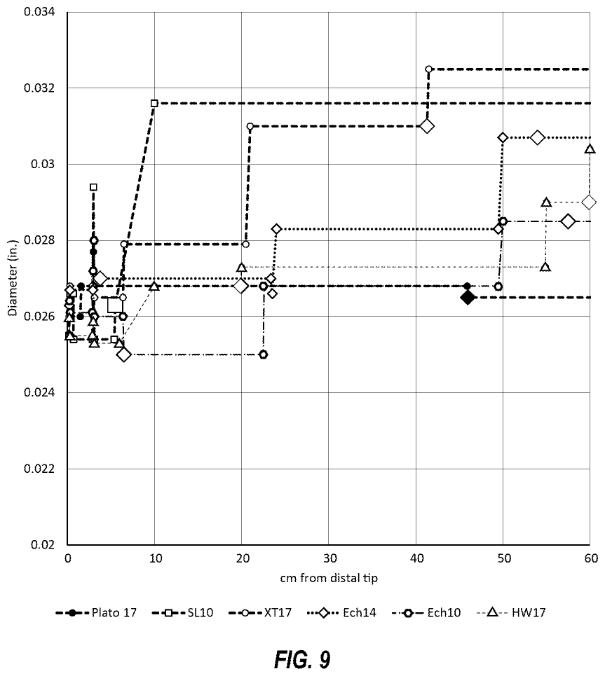

FIG. 9 compares outer diameters along distal lengths of the Plato 17 device according to the present disclosure to various conventional catheter devices. The larger data points represent points where a polymer transition is visibly apparent. The data from FIG. 9 is also represented in Table 2, which shows the maximum diameter change within the distal 15 cm section and the distal 35 cm section of each of the catheter devices. As shown, the Plato 17 diameter changes by no more than 0.0017 inches across the distal 15 cm section and across the distal 35 cm section.

TABLE 2
Diameter Changes of Various Microcatheters Across
Different Distal Section Sizes.

| | |
|---|---|
| Section Measured from | Maximum Diameter Change |
| Distal End (cm) | (inches) |

Plato 17

| | |
|---|---|
| 0-15 | .0017 |
| 0-35 | .0017 |

SL-10

| | |
|---|---|
| 0-15 | .0062 |
| 0-35 | .0062 |

XT-17

| | |
|---|---|
| 0-15 | .0024 |
| 0-35 | .0055 |

Echelon-14

| | |
|---|---|
| 0-15 | .0017 |
| 0-35 | .0020 |

Echelon-10

| | |
|---|---|
| 0-15 | .0022 |
| 0-35 | .0022 |

Headway-17

| | |
|---|---|
| 0-15 | .0015 |
| 0-35 | .0020 |

In some embodiments, a catheter device as described herein has (1) a bending stiffness of 5×10⁻⁶ N·m² or greater in at least a portion of the distal 35 cm of the catheter device, (2) a change in outer diameter of no more than 0.002 inches across the distal 15 cm and/or distal 35 cm section, and (3) a bending stiffness slope ((N·m²)/cm)) of no more than about 1.3×10⁻⁶ for a distal 15 cm section, no more than about 4.2×10⁻⁶ for a distal 35 cm section, and/or no more than about 1.1×10⁻⁵ for a distal 50 cm section. While the Plato 17 device configured according to the present disclosure achieved each of the foregoing features, none of the other prior catheter devices tested were able to do so. That is, the Headway-17 catheter does not meet the minimum bending stiffness requirement, the Echelon-10 does meet the diameter change requirement, and none of the other tested catheters meet the slope requirements.

In some embodiments, the beneficial bending stiffness profile features described above are specifically applicable to a transition section where a first polymer of the outer member transitions to a second polymer of the outer member.

In some embodiments, the shaft 112 maintains substantially the same wall thickness along its length. Other catheter devices based on coils and/or braids will most often have adjusted wall thicknesses at transition points. Changes in wall thickness can introduce additional kink or stress points and/or require additional manufacturing steps to manage.

In some embodiments, there may be an acceptable bending stiffness change associated with the distal tip region because the shaft 112 transitions to the coil 114 and/or the coil 114 transitions to the distal-most section of liner 110. These stiffness changes may be acceptable because they are so near the distal end 103. Thus, in some embodiments, the distal-most 3 to 5 cm may be excepted from the foregoing bending stiffness change limits.

### Fatigue Resistance

The catheter devices described herein also beneficially provide effective fatigue resistance. For example, in a bend and twist fatigue test method based on ASTM E2948, catheter devices as described herein are capable of achieving greater than 20 cycles before breaking. The bend and twist fatigue test method is described in more detail in document TM-00127, which is attached hereto as Appendix 1. In short, the test is adapted from ASTM E2948, which is a standard test method for measuring rotating bending fatigue of solid round fine wire.

The effective fatigue resistance of the disclosed catheter devices may be present along the entire length of the shaft portion of the device, or along one or more sub-sections of the device (e.g., along one or more sections having length of about 3 to 35 cm, or about 3 to 20 cm, or about 3 to 10 cm). The effective fatigue resistance is provided by one or more of the following parameters: (1) maintaining ring widths at less than or equal to about 30% of the corresponding outer diameter of the shaft 112; and/or (2) maintaining cut depths at greater than or equal to about 11% of the outer diameter of the shaft 112.

### ADDITIONAL TERMS & DEFINITIONS

While certain embodiments of the present disclosure have been described in detail, with reference to specific configurations, parameters, components, elements, etcetera, the descriptions are illustrative and are not to be construed as limiting the scope of the claimed invention.

As used herein, the term “microfabricated” refers to any fabrication process capable of manipulating a stock material to form a catheter device having one or more of the features disclosed herein, including any fabrication process capable of forming gaps in an inner shaft as disclosed herein. Examples include, but are not limited to, laser cutting and blade cutting.

For any given element of component of a described embodiment, any of the possible alternatives listed for that element or component may generally be used individually or in combination with one another, unless implicitly or explicitly stated otherwise.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A catheter device, comprising:
an inner shaft having a plurality of gaps formed therein;
an outer member comprising a polymer material disposed within the gaps, wherein the catheter device provides non-linear bending stiffness such that bending becomes more difficult as the bend angle increases;
a distal radiopaque marker band; and
a proximal radiopaque marker band,
wherein the proximal marker band is positioned on the shaft at a groove formed into the circumference of the shaft.

2. The catheter device of claim 1, wherein the inner shaft includes a plurality of axially extending beams that couple a plurality of circumferentially extending rings.

3. The catheter device of claim 2, wherein the axially extending beams extend parallel to a longitudinal axis of the tube structure.

4. The catheter device of claim 2, wherein the circumferentially extending rings form fully enclosed rings.

5. The catheter device of claim 1, wherein the outer member includes multiple different polymer durometers.

6. The catheter device of claim 5, wherein the outer member includes a transition section where a first polymer is adjacent to a second polymer of different hardness, the transition section including a change in bending stiffness of the outer member, and wherein the microfabricated shaft includes a section coincident with the transition section that is configured to compensate for the change in bending stiffness of the outer member.

7. The catheter device of claim 6, wherein the second polymer proximal of the first polymer and has a greater hardness than the first polymer such that the outer member increases in bending stiffness across the transition section in the distal to proximal direction.

8. The catheter device of claim 6, wherein the shaft does not increase in bending stiffness across at least a portion of the transition section to compensate for the increase in bending stiffness of the outer member.

9. The catheter device of claim 6, wherein the shaft changes in bending stiffness because of one or more of: a change in spacing between cuts; a change in depth of cuts; a change in spacing of the cuts, or a change in number of beams connecting adjacent pairs of rings.

10. The catheter device of claim 1, further comprising an inner liner around which the shaft is positioned.

11. The catheter device of claim 10, wherein the polymer material of the outer member is fused to the liner, fills the gaps of the shaft, and covers outer surfaces of the shaft to encapsulate and embed the shaft.

12. The catheter device of claim 1, wherein the outer material comprises one or more polyether block amide polymers.

13. The catheter device of claim 1, wherein the catheter device has a bending stiffness slope ((N·m²)/cm)) of no more than about 6.0×10⁻⁷ for a distal 15 cm section, no more than about 9.0×10⁻⁷ for a distal 35 cm section, and/or no more than about 9.0×10⁻⁷ for a distal 50 cm section.

14. The catheter device of claim 1, wherein at least a portion of the distal 35 cm of the catheter device has a bending stiffness of 5×10⁻⁶ N·m² or greater, and wherein the catheter device has a bending stiffness slope ((N·m²)/cm)) of no more than about 4.0×10⁻⁶ for a distal 35 cm section and/or no more than about 4.5×10⁻⁶ for a distal 50 cm section.

15. The catheter device of claim 1, wherein: (1) at least a portion of the distal 35 cm of the catheter device has a bending stiffness of 5×10⁻⁶ N·m² or greater; (2) the catheter device has a change in outer diameter of no more than 0.002 inches across the distal 15 cm and/or distal 35 cm section; and (3) the catheter device has a bending stiffness slope ((N·m²)/cm)) of no more than about 1.3×10⁻⁶ for a distal 15 cm section, no more than about 4.2×10⁻⁶ for a distal 35 cm section, and/or no more than about 1.1×10⁻⁵ for a distal 50 cm section.

16. The catheter device of claim 1, further comprising a coil disposed distal of the shaft.

17. The catheter device of claim 16, wherein the coil has a variable pitch.

18. The catheter device of claim 17, wherein one or both ends of the coil includes a region of narrowed pitch relative to an intermediate portion of the coil.

19. A catheter device, comprising:
an inner shaft having a plurality of gaps formed therein;
an outer member comprising a polymer material disposed within the gaps, wherein the catheter device provides non-linear bending stiffness such that bending becomes more difficult as the bend angle increases;
a coil disposed distal of the shaft;
a distal radiopaque marker band disposed distal of the coil; and
a proximal radiopaque marker band disposed on the shaft proximal of the coil,
wherein the proximal marker band is positioned on the shaft at a groove formed into the circumference of the shaft.

20. The catheter device of claim 19, wherein the coil has a variable pitch.
